(19) United States
(12) Patent Application Publication
(10) Pub. No.: US 2018/0012359 A1
(43) Pub. Date: Jan. 11, 2018

(54) Systems and Methods for Automated Image Classification and Segmentation

(71) Applicants: Pavle Prentasic, Sokolovac (HR); Morgan Lindsay Heisler, Maple Ridge (CA); Sven Loncaric, Zagreb (HR); Marinko Venci Sarunic, Burnaby (CA); Mirza Faisal Beg, Coquitlam (CA); Sieun Lee, Vancouver (CA); Andrew Brian Merkur, Vancouver (CA); Eduardo Navajas, Vancouver (CA); Zaid Mammo, Vancouver (CA)

(72) Inventors: Pavle Prentasic, Sokolovac (HR); Morgan Lindsay Heisler, Maple Ridge (CA); Sven Loncaric, Zagreb (HR); Marinko Venci Sarunic, Burnaby (CA); Mirza Faisal Beg, Coquitlam (CA); Sieun Lee, Vancouver (CA); Andrew Brian Merkur, Vancouver (CA); Eduardo Navajas, Vancouver (CA); Zaid Mammo, Vancouver (CA)

(73) Assignee: Marinko Venci Sarunic, Burnaby, BC (CA)

(21) Appl. No.: 15/642,290

(22) Filed: Jul. 5, 2017

Related U.S. Application Data
(60) Provisional application No. 62/358,573, filed on Jul. 6, 2016.

(51) Int. Cl. G06T 7/00; G06N 3/04; A61B 3/14; G06K 9/00

(52) U.S. Cl. CPC G06T 7/0014; G06N 3/0427; G06T 7/11; A61B 3/14; G06K 9/0061; G06K 9/00597

(57) ABSTRACT

Optical coherence tomography (OCT) may be used to acquire cross-sectional or volumetric images of any specimen, including biological specimens such as the retina. Additional processing of the OCT data may be performed to generate images of features of interest. In some embodiments, these features may be in motion relative to their surroundings, e.g., blood in the retinal vasculature. The proposed invention describes a combination of images acquired by OCT, manual segmentations of these images by experts, and an artificial neural network for the automated segmentation and classification of features in the OCT images. As a specific example, the performance of the systems and methods described herein are presented for the automatic segmentation of blood vessels in images acquired with OCT angiography.

### CROSS REFERENCE TO RELATED APPLICATIONS

The instant application is a utility application and claims priority to the pending US provisional patent application: 62/358,573 titled “Segmentation of the Retinal Microvasculature using Deep Learning Networks.”, filed on the 6 Jul. 2016. The entire disclosure of the Provisional U.S. patent Application No. 62/358,573 is hereby incorporated by this reference in its entirety for all of its teachings. This benefit is claimed under 35. U. S. C. $119.

### FIELD OF TECHNOLOGY

The description is relevant to imaging of biological specimen such as a retina using a form of low coherence interferometry such as optical coherence tomography (OCT) and optical coherence domain reflectometry (OCDR). Embodiments of this invention relate to automated identification of features in these images, such as blood vessels.

### BACKGROUND

Optical coherence tomography (OCT) provides cross-sectional images of any specimen including biological specimens such as the retina with exquisite axial resolution, and is commonly used in ophthalmology. OCT imaging is an important aspect of clinical care. In ophthalmology, it is used to non-invasively visualize the various retinal structures to aid in better understanding of the pathogenesis of vision-robbing diseases.

Extensions to conventional OCT imaging have been developed for enhancing visualization of the blood circulation, also referred to as angiography. The resulting image data is information rich, and requires proper analysis to assist with screening, diagnosis, and monitoring of retinal diseases.

Previously reported approaches to segmentation of the blood vessels in OCT Angiography (OCT-A) relied on intensity thresholding. Although this approach is quick and easy to implement, the quality of the segmentation results depends on the contrast of the vessels, and background noise levels. Manual segmentation of the retinal blood vessels in OCT-A images, which is the current gold standard, is a time-consuming and tedious task which requires training by experts to accurately identify the features of interest from the noise. Accurately automating the segmentation of these vessels is paramount to creating a useful output in an expedient manner. A limitation of manual segmentation is that it suffers from inter-rater differences, particularly for low contrast features. Even the same rater performing manual segmentation of the same image at different times produces different results (intra-rater variation), particularly for low contrast features.

### SUMMARY

The invention discloses a system and method for automated segmentation and classification of OCT images. In one embodiment, the OCT system comprises a beam splitter dividing the beam into sample and reference paths, light delivery optics for reference and sample, a beam combiner to generate optical interference, a detector to convert the signal into electronically readable form, and a processor for controlling the acquisition of the interference signal and generating images. In one embodiment of the invention, the OCT system is configured for the acquisition of retinal images with blood vessels and capillaries emphasized; such images are referred to as angiograms. In one embodiment of the invention, each pixel of at least one OCT image, and preferably a plurality of OCT images, is manually segmented by at least one expert as either being a part of a feature, or belonging to the background. In one embodiment, the features are the retinal layers. In another embodiment, the features are fluid in the retina. In another embodiment, the features are the blood vessels and capillaries. In another embodiment, the features are lymph vessels. The manually segmented images are used to train an artificial neural network to automatically extract the features from new images. In some embodiments, new images are segmented using the trained neural network to extract vessels.

The parameters of the artificial neural network are determined using OCT images that have been manually segmented by experts. The training set may be generated by a single expert, or may be made more robust through the inclusion of multiple examples of segmentations from different raters, and repeat segmentations by the same raters. The training set may be performed by expert manual correction of automatic segmentations performed using various methods. Some methods may be coarse or inaccurate methods.

Representative results of an embodiment of the invention demonstrate the high effectiveness of the deep learning approach to replicate the segmentation of blood vessels in OCT-A images by medical experts with an artificial neural network. In one embodiment, the specimen could be a retina or a choroid or a finger or any other part of the body. For the purposes of assisting the explanation, the results of using an artificial neural network to segment images acquired from a clinical prototype OCT-A system were compared to the manual segmentations from two separate trained raters as a demonstration.

### BRIEF DESCRIPTION OF THE DRAWINGS

The following drawings will be used to assist in the description of the invention by way of example only, and without disclaimer of other embodiments.

FIG. 1A shows a schematic representation of an OCT system used for imaging the eye.

FIG. 1B shows a schematic representation of a generalized. OCT system.

FIG. 2A is a flow chart of a method for creating a neural network for automatically segmenting vasculature. This is an embodiment of the invention.

FIG. 2B is a generalized flow chart of FIG. 2A for creating a neural network for automatically segmenting features which covers more embodiments of the invention.

FIG. 3 is a graphical representation of a network structure. This is an embodiment of the invention.

FIG. 4 is an example of the original image, manual segmentation, output, and binarized output from one embodiment of the invention.

FIG. 5 shows the accuracy of the segmentation using the example as one embodiment of the invention.

FIG. 6 shows the mean accuracy of segmentation using the example as one embodiment of the invention.

FIG. 7 shows the receiver operating characteristic (ROC) using the example as one embodiment of the invention.

FIG. 8 shows the F1 measure, which is another measurement of the accuracy, using the example as one embodiment of the invention.

FIG. 9 is a table of mean capillary density comparison between Rater A1, Rater A2, Rater B, and the network.

### DETAILED DESCRIPTION

This invention describes a novel approach to the segmentation of various features in OCT images including blood vessels in OCT-A images.

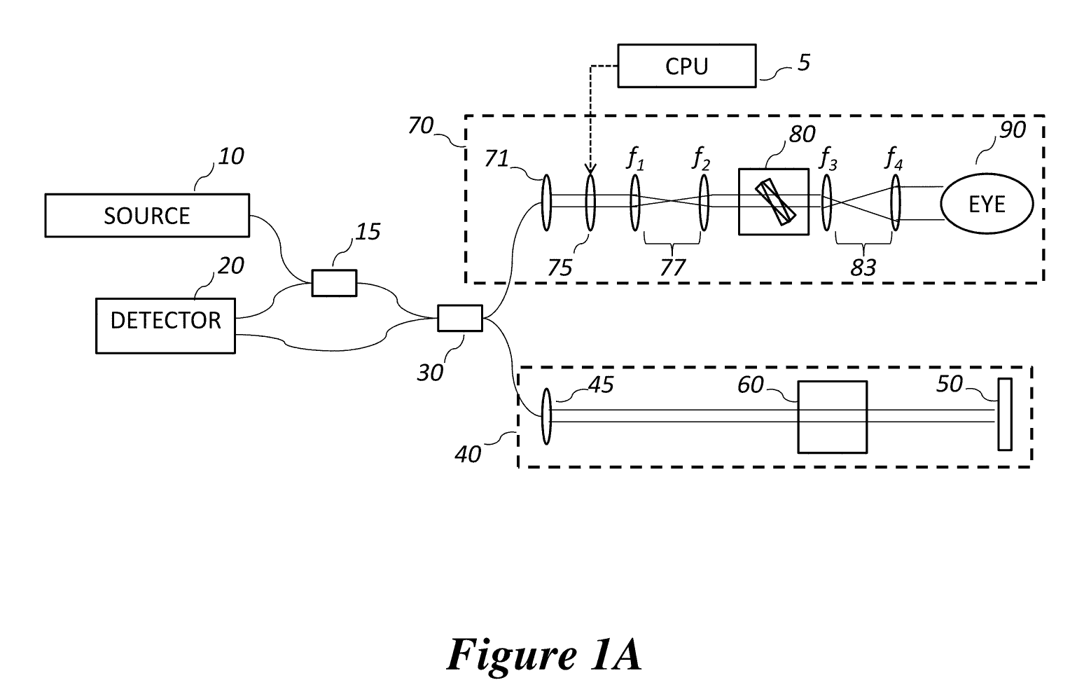

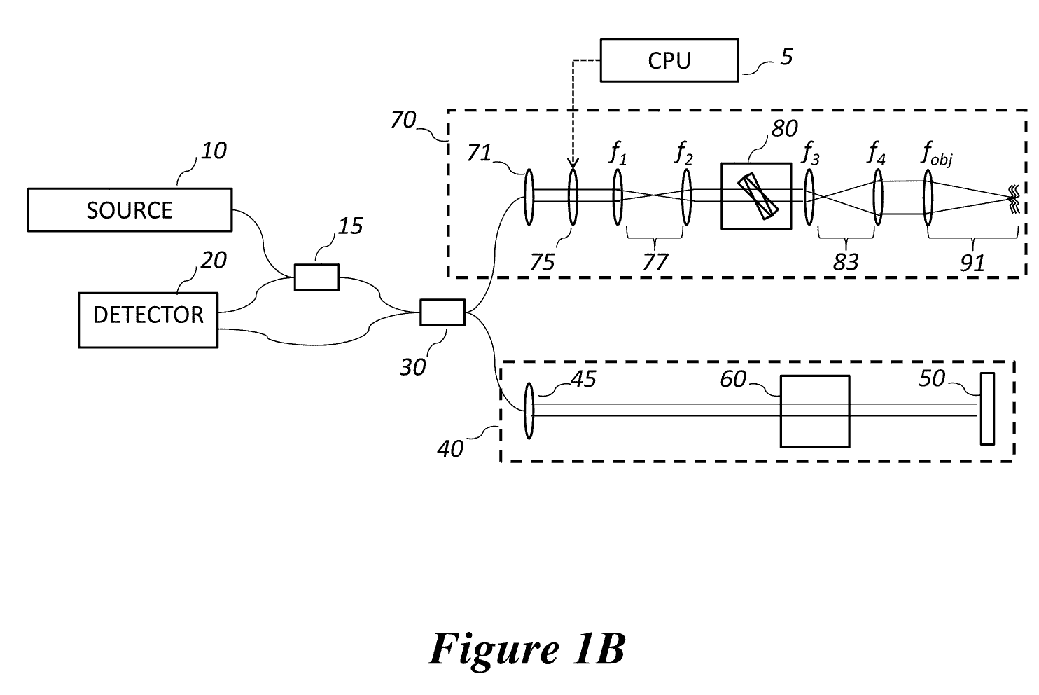

A demonstration of the invention was performed on a custom developed prototype OCT or OCT-A acquisition system. Examples of representative OCT embodiments are presented in FIG. 1A and FIG. 1B. In the demonstrated configuration, the OCT engine is based on a wavelength swept laser (or light source) (10), in a configuration known as Swept Source (SS) OCT or alternatively as Optical Frequency Domain Imaging (OFDI). The detector is a balanced photodiode (20). An unbalanced photodiode could also be used. The OCT system is computer controlled, combining and providing signals for timing and control, and for processing the interferometric data into images or volumetric data. It could be controlled using an embedded controller as well. The fibre coupler (30) splits the light from the source into reference (40) and sample (70) paths or arms. The fibre coupler may have a splitting ratio of 50/50, or some other ratio. In some embodiments, the fibre coupler (or fibre optic beam splitter) is replaced by a free-space beam splitter. The reference arm has a mirror (50) typically mounted on a translation stage and may contain dispersion compensating optical elements (60). In an alternate embodiment, the reference arm could be a fibre with a fibre-integrated mirror. In one embodiment, shown in FIG. 1A, the sample arm optics (70) are designed for high resolution imaging of a retina, and the final objective is the cornea and intraocular lens of the eye (90). Alternately, in another embodiment shown in FIG. 1B, the OCT system could be used to image another type of sample, in which case an objective lens would be useful before the sample in the optical setup (91). A scanning mechanism (80) is used to scan the angle of light incident on the cornea, which in turn scans the lateral position of the focused spot on the retina. In another embodiment, the sample arm optics (70) are designed for high resolution imaging of a specimen. The light returning from the sample and reference arms are combined through the beam splitter to create an interference signal and directed towards the detector (20). The optical interference signal is processed to construct a depth resolved image or images of the sample. The control of the scanning mechanism in the sample arm can be used to acquire three dimensional (3D) volumetric data of the sample. In some embodiments, the sample could be a human or animal eye.

Alternative variations of this configuration could replace the SS OCT with a Spectral Domain/Spectrometer Domain (SD) OCT or a Time Domain (TD) OCT. For Spectral Domain OCT, the swept laser is replaced by a broad band light source, and the detector is spectrally resolved, for example, using a spectrometer. For Time Domain OCT, the swept laser is replaced by a broad band light source, and the reference minor position is scanned axially (or angularly) to generate interference fringes. Thus, TD-OCT comprises of a scanning reference mirror; wherein the reference mirror is scanned to modulate an optical path-length in the reference arm. Operating wavelengths for retinal imaging are from the visible to near infrared. In one embodiment, the central wavelength is 1060 nm, with a bandwidth of ˜70 nm. In another embodiment, the central wavelength is 840 nm, with a bandwidth of ˜50 nm. Other embodiments may use combinations of central wavelength ranging from 400 nm to 1300 mn, and bandwidth of approximately 5 nm up to over 100 nm, and in some cases with central wavelengths around 700 nm and bandwidths of several 100's of nanometers. In other embodiments, higher or lower source wavelengths could be used. In some embodiments the fibre coupler (15) is an optical circulator. In other embodiments of the system, the detection may not be balanced, and fibre coupler (15) may be replaced by a direct optical path from the source to the interferometer fibre coupler (30). Alternative variations of the interferometer configuration could be used without changing the imaging function of the OCT engine.

The instrument control sub-system may further comprise of at least one processor configured to provide timing and control signals; at least one processor for converting the optical interference to the 1 or 2 or 3-dimensional data sets. The processor(s) may extract a specific depth layer image from the three dimensional data sets, or a range of depth layer images. In one embodiment, the depth layers are summed along the axial direction to generate a 2D image. Other approaches of combining the axial depth information in the range of depth layers include maximum intensity projection, median intensity projection, average intensity projection, and related methods. A two dimension (2D) depth layer extracted from a 3-D volume is called an en face image, or alternatively in some embodiments, a C-scan.

At each scan position, corresponding to a point on the specimen, the OCT signal acquired at the detector is generated from the interference of light returning from the sample and the reference arms.

In some embodiments, a common path OCT system may be used; in this configuration, a reference reflection is incorporated into the sample arm. An independent reference arm is not needed in this embodiment. Light from the source is directed into a 3 or 4 port beam splitter, which in some embodiments is a bulk optic beam splitter or a 2×1 fiber splitter, or which in other embodiments is an optical circulator. Light in the source arm is directed by the beam splitter to the sample arm of the common path interferometer. A reference reflecting surface is incorporated into the sample arm optics, at a location that is within the interference range of the sample. In some embodiments, the reference reflection may be located approximately integer multiples of the light source cavity length away from the sample, utilizing ‘coherence revival’ to generate interference. Light returning from the sample and light returning from the reference reflecting surface are directed back through the beam splitting element toward the detector arm, generating interference fringes as with conventional OCT systems. The interference is digitized, and the remaining OCT processing steps are similar as with conventional interferometric configurations. By having a reference reflection integrated with the sample, common path interferometry is immune to phase fluctuations that may be problematic in conventional interferometers with two, or more, arms. In the case of using an optical circulator in the place of a 2×1 coupler, the efficiency is higher. The source and detector combination may comprise a spectral domain OCT system, or a swept source OCT system.

The interference signal is processed to generate a depth profile of the sample at that position on the specimen, called an A-scan. A cross-sectional B-scan image is generated by controlling the scanning of the position of the beam laterally across the specimen and acquiring a plurality of A-scans; hence a two dimensional (2D) B-scan depicts the axial depth of the sample along one dimension, and the lateral position on the sample in the other dimension. A plurality of B-scans collected at the same location is referred to as a BM-scan (a collection of B-scans acquired in M-mode fashion). A collection of BM-scans acquired at different locations on the retina constitute a volume. In one embodiment, the BM-scans are acquired at different lateral positions on the sample in a raster-scan fashion. In another embodiment, the BM-scans are acquired in a radially spoked pattern. In other embodiments, the BM-scans are acquired in a spiral, or Lissajous, or other patterns, in order to acquire a volumetric image of the sample.

In another embodiment, the OCT system may be ‘full field’, which does not scan a focused beam of light across the sample, but rather uses a multi-element detector (or a 1-dimensional or 2 dimensional detector array) in order to acquire all lateral positions simultaneously.

In another embodiment, the OCT interferometer may be implemented in free space. In a free space interferometer, a conventional bulk optic beam splitter (instead of the fiber-optic splitter (30)) is used to divide the light from the source into sample and reference arms. The light emerging from the bulk optic beam splitter travels in free space (i.e., air), and is not confined in a fiber optic, or other form of waveguide. The light returning from the sample and reference arms are recombined at the bulk optic beam splitter (working as a beam combiner in the reverse direction) and directed toward at least one detector. The interference between the light returning from the sample arm, and the light returning from the reference arm is recorded by the detector and the remaining OCT processing steps are the same as with fibre based interferometric configurations. The source and detector combination may comprise a spectral domain OCT system, or a swept source OCT system, or a time domain OCT system.

In some embodiments, the back-scattering intensity contrast of the blood vessels relative to the surrounding tissue may provide adequate contrast for visualization of the retinal blood vessels. In one embodiment, the increased contrast from the blood vessels may arise due to high resolution retinal imaging. In one embodiment, the increased lateral resolution is achieved using a larger diameter beam incident on the cornea. In one embodiment, the beam diameter may be greater than 2.5 mm. In another embodiment, the increased lateral resolution is accompanied by adaptive optics in order to achieve a diffraction limited, or close to diffraction limited, focal spot at the retina. In one embodiment, the adaptive optics will comprise an optical element to shape the wavefront of the incident beam of light, such as a deformable mirror, deformable lens, liquid crystal, digital micromirror display, or other spatial light modulator. In one configuration, the shape of the wavefront controlling element is determined using a wavefront sensor. In another embodiment, a sensorless adaptive optics method and system may be used, in which a merit function is calculated on the image quality in order to control the wavefront shaping optical element. More detailed information on sensorless adaptive optics may be found at the following reference: Y. Jian, S. Lee, M. J. Ju, M. Heisler, W. Ding, R. J. Zawadzki, S. Bonora, M. V. Sarunic, “Lens-based wavefront sensorless adaptive optics swept source OCT,” Scientific Reports 6, 27620 (2016).

In one embodiment, the parameters of the OCT acquisition system and the parameters of the processing methods are used to enhance the contrast of flowing material; this system and method is referred to as OCT-Angiography (OCT-A). In one embodiment, the features of interest in OCT-A are blood vessels or capillaries, which may be visualized in B-scans, or en face images, or in the 3D OCT volumetric datasets. OCT images with blood vessel or capillary contrast are referred to as angiograms. Flow contrast to enhance the appearance of the blood vessels in the angiograms may be performed using any number of methods.

Comparison of the difference or variation of the OCT signal between B-scans in a BM-scan on a pixel-wise basis enhances the contrast of the blood flow in the vessels relative to the static retinal tissue.

In one embodiment of flow contrast enhancement of blood vessels, called speckle variance (sv) OCT, the pixel-wise comparison is performed by calculating the variance of the intensity values at the corresponding pixels in each B-scan of a BM-scan. An example of the equation used to compute each speckle variance frame (svⱼₖ), from the intensity data from the BM-scans in an OCT volume (Iᵢⱼₖ) is

$${sv}_{jk} = \frac{1}{N} {\sum }_{i = 1}^{N} {( {I}_{ijk} - \frac{1}{N} {\sum }_{i = 1}^{N} {I}_{ijk} )}^{2} ,$$

where I is the intensity of a pixel at location i,j,k; i is the index of the B-scan frame; j and k are the width and axial indices of the i^{th} B-scan; and N is the number of B-scans per BM-scan. In one embodiment, the volume size is j=1024 pixels per A-scan, k=300 A-scans per B-scan, and N=3 for a total of 900 B-scans (300 BM-scans) per volume.

In another embodiment of flow contrast enhancement called phase variance (pv) OCT, the calculation utilizes the phase of the OCT signal or the optical interference signal. In another embodiment, the calculation utilizes the complex OCT signal. In other embodiments, the number of B-scans per BM-scan may be as low as 2, or greater than 2. In other embodiments, the OCT A-scan in the spectral domain may be divided into multiple spectral bands prior to transformation into the spatial domain in order to generate multiple OCT volume images, each with lower axial resolution than the original, but with independent speckle characteristics relative to the images reconstructed from the other spectral bands; combination of the flow contrast signals from these spectral sub-volumes may be performed in order to further enhance the flow contrast relative to background noise. Other embodiments of flow contrast to enhance the appearance of blood vessels may include optical microangiography or split spectrum amplitude decorrelation angiography, which are variations of the above mentioned techniques for flow contrast.

In another embodiment of flow contrast, the spatial oversampling may be used to detect motion on an A-scan basis. This approach is referred to as Doppler OCT. In this configuration, the change in phase of the OCT signal at adjacent (and overlapping) A-scans is used to determine flow.

Following acquisition with an imaging device, the images are stored on an electronically readable medium. In one embodiment, the imaging device is an OCT system. At least one image and preferable a plurality of images, are manually segmented by experts to label features of interest. The experts may be clinicians, scientists, engineers, or others with specific training and with the segmentations reviewed by a person or persons of professional authority. The images that are segmented may be cross-sectional images (B-scan) or en face images. 3-D datasets could also be used instead of images for segmenting the features of interest. The features (of interest) to be segmented may be layers, regions of fluid, regions of swelling, lymph vessels, blood vessels, or capillaries, or regions of new blood vessel and capillary growth. The segmented images may be stored on an electronically readable medium. The original images and the manual segmentations are used as inputs to train an artificial neural network to extract the features of interest. In one embodiment, the artificial neural network may be a convolutional neural network, or a deep convolutional neural network. For the purposes of training the parameters of the network, the artificial neural network may be implemented in software on a general purpose processor, such as a central processing unit (CPU), graphics processing unit (GPU), a digital signal processor (DSP) or reduced instruction set computer (RISC) processor, or related. Once trained, the artificial neural network is used to segment these features on newly acquired images that were not a part of the training set. In one embodiment, after training; the artificial neural network is implemented in at least one, or a combination of: software on a general purpose processor, such as a CPU, GPU, RISC, or related; or hardware, such as a Field Programmable Gate Array (FPGA), or application specific integrated circuit (ASIC) or a digital signal processor (DSP) hardware.

In one embodiment, the processor of the OCT system generates images comprising flowing material. The features of interest can be capillaries and/or vessels. In another embodiment, the processor generates angiograms.

Thus, the image generated could be an en-face image, or an angiogram or an en-face angiogram. In one embodiment, the angiograms are superimposed on en-face images. In another embodiment, segmentation results are super-imposed on en-face images.

In one embodiment, for the purpose of demonstration of the invention, OCT-A images were acquired from the foveal region in 12 eyes from 6 healthy volunteers aged 36.8±7.1 years using a GPU-accelerated OCT or OCT-A clinical prototype. In total, 80 images were acquired and used for this demonstration. In this embodiment, the scan area was sampled in a 300×300(×3) grid with a ˜1×1 mm field of view in 3.15 seconds. In other embodiments, the size of the scan area, the sampling density, or number of B-scans per BM-scan may be changed. Without loss of generality, in other embodiments, the scan area may be in the range of <1 mm to >10 mm, the sampling dimensions may be in the range of 20 to 10,000, or larger, per scan dimension, or the number of B-scans per BM-scan may be between 2 and 10, or larger.

In other embodiments, the retinal images may be acquired by a retinal fundus camera, Scanning Laser Ophthalmoscopy, Photo-Acoustic Microscopy, laser speckle imaging, retinal tomography (e.g., Heidelberg Retinal Tomography), retinal thickness analyzer, or other technique that provides adequate contrast of the blood vessels. With any of these techniques, contrast agents may be used to enhance the visibility of the vessels, for example, fluorescence retinal angiography using a retinal fundus camera. One may use contrast agents such Fluorescein and Indocyanine Green (ICG) to enhance the contrast of blood vessels.

Ground truth segmentations of the training set of images are required to train the neural network. The ground truth segmentations represent the knowledge base of the experts including clinical experts on retinal blood vessel anatomy. In one embodiment, expert raters manually segmented the OCT-A images using a Wacom Intuos 4 tablet and GNU Image Manipulation Program. In other embodiments, segmentations of the vessels for training the dataset may be performed with one or more raters, or using different methods, or validated by expert raters. The ground truth segmentations are saved and paired with the original image data for training the automated method to reproduce the knowledge base of the experts.

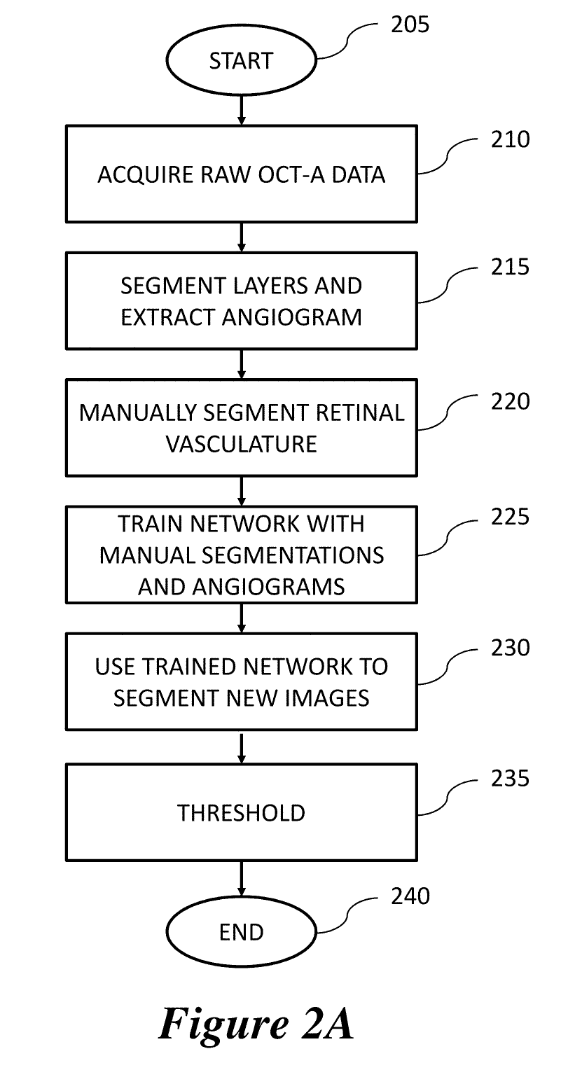

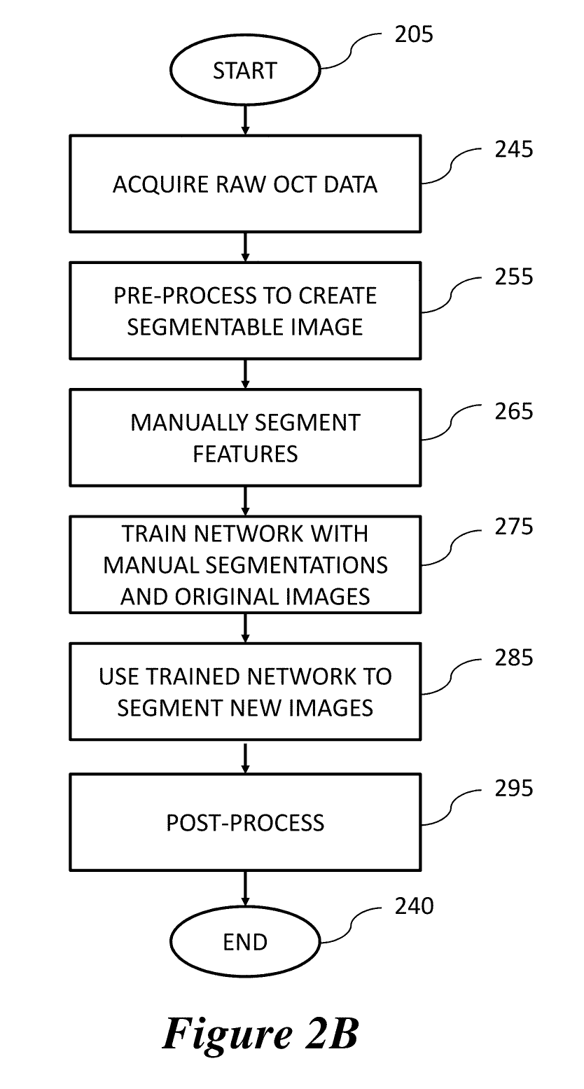

FIG. 2A represents a high level flow chart for one embodiment of the invention. To start (205) OCT-A data is acquired (210) with an OCT system, possible embodiments of which are shown in FIG. 1A and FIG. 1B. The retinal vessels are contained in specific cell layers of the retina. In order to extract the portion of the retina containing the vessels, the retinal layers are segmented (215); in one embodiment, the retinal layers can be segmented using a graph-cut based segmentation method. In another embodiment, the retinal layers are segmented manually. In another embodiment, the retina layers are segmented through a combination of automated methods and manual delineation of low contrast features, or manual correction of the automated segmentation. The retinal angiogram is then created from the desired retinal layers. In one embodiment, all of the vascular layers may be extracted from the OCT volume, and the retinal angiogram are generated by summing along the axial direction. Other approaches of combining the axial depth information of the blood vessels include maximum intensity projection, median intensity projection, mean intensity projection and related methods. In other embodiments, only one or a subset of the retinal layers may be extracted to generate the retinal angiograms. Manual raters then segment the vasculature in the chosen angiograms (220). The segmentations and the retinal angiograms are used as inputs for training a neural network (225). In one embodiment, the neural network may be deep convolutional neural network or a convolutional neural network. In one embodiment, the training may be performed on a Graphics Processing Unit (GPU), or on a central processing unit (CPU). The trained network can then be used to segment new angiograms (230). In one embodiment, the network outputs a probability map, where each pixel is classified by the probability that it is a vessel. This output can then be thresholded (235) using Otsu's method or other similar algorithm to binarize the segmentation, which concludes the algorithm (240). The trained network may be transferred to hardware different than that used for training. The trained network may be implemented on a central processing unit (CPU), application specific integrated circuit (ASIC), digital signal processor (DSP), GPU, or Field Programmable Gate Array (FPGA), or related hardware.

FIG. 2B represents a more general case of FIG. 2A which encompasses more embodiments of the invention. To start (205) OCT data is acquired (245) with an OCT system, an embodiment of which is shown in FIG. 1A and FIG. 1B. Then, data is extracted from the volumetric OCT data and pre-processed into an image for segmentation (255). In one embodiment, this could be a cross-sectional scan of the specimen, or en face images. The images may contain normal or pathological features to be segmented. The features are segmented from the extracted OCT image by an expert (265). The segmentations may be performed fully manually, or expert manual corrections of a course segmentation performed by some other automated method. The segmentations and extracted OCT images are used as inputs for training an artificial neural network (275). In one embodiment, the artificial neural network may be deep convolutional neural network or a convolutional neural network. In one embodiment, the training may be performed on a Graphics Processing Unit (GPU), or on a central processing unit (CPU). The trained network can then be used to segment new images (285). This output can then be post-processed (295) in order to provide a clinically useful output, which concludes the algorithm (240). The trained network may be transferred to hardware different than that used for training. The trained network may be implemented on a central processing unit (CPU), application specific integrated circuit (ASIC), digital signal processor (DSP), GPU, or Field Programmable Gate Array (FPGA), or related hardware.

The automated segmentation of the blood vessels in the OCT-A images was performed by classifying each pixel into vessel or non-vessel class using a deep convolutional neural network. In one embodiment of the invention, convolutional layers and max pooling layers are used as hierarchical feature extractors, which map raw pixel intensities into a feature vector. The feature vector describes the input image, which is then classified using fully connected layers.

The convolutional layers are made of a sequence of square filters, which perform a 2D convolution with the input image. The convolutional responses are summed and passed through a nonlinear activation function. In one embodiment, the nonlinear activation function is a rectifying linear unit, which implements the function f(x)=max(0, x). In other embodiments, other nonlinear (or linear) activation functions could be used. Multiple maps are used at each layer to capture different features from the input images to be used for classification.

The max pooling layers generate their output by taking the maximum value of the activation over non-overlapping square regions. By taking the maximum value of the activation function, the most prominent features are selected from the input image. In one embodiment, layers do not have adjustable parameters and their size is fixed.

Drop out layers may be used to reduce overfitting of the network during training. A drop out layer reduces the number of connections by removing them probabilistically. The purpose of the drop out layer is to prevent network over-fitting, and to provide a way of combining a plurality of neural networks in an efficient manner. Drop out may be alternatively implemented by using a drop out at each connection, or stochastic pooling, or other methods.

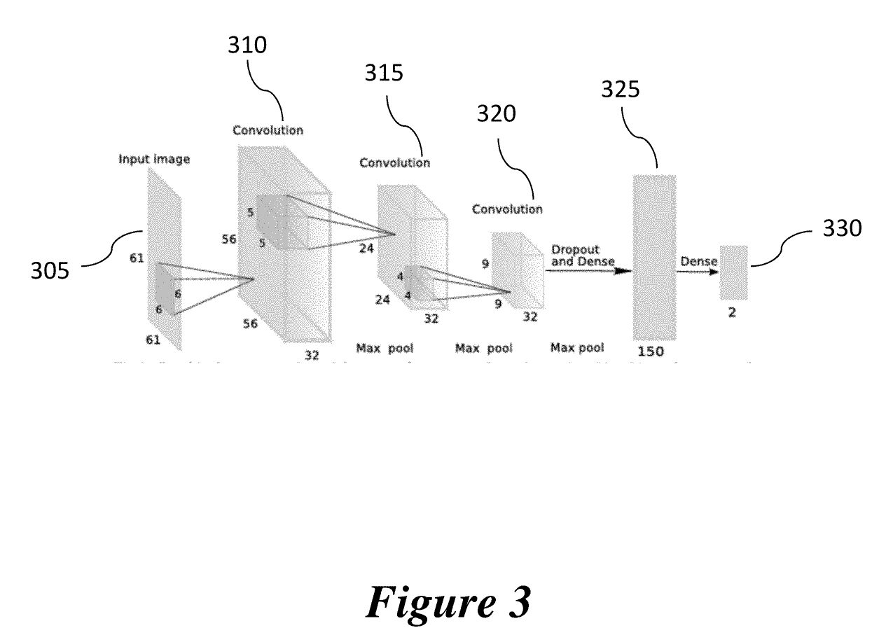

One embodiment of a neural network architecture for retinal vessel segmentation is presented graphically in FIG. 3 for the purpose of example only (and not by limitation). Each training example comprises of a 61×61 pixel square window around the training pixel (305). After six stages of varied convolutional and max pooling layers (310-320), a dropout layer is inserted (325). Then, two fully connected layers are used to classify the feature vector generated by the previous layers. The final fully connected layer contains two neurons where one neuron represents the vessel class and other neuron represents the non-vessel class (330).

In this implementation of the deep convolutional neural network, six layers of convolutional and max pooling layers with varying parameters are used. The number of layers may be varied without a loss of generality. In one embodiment, 32 feature maps are used at each layer. The number of maps may be varied without a loss of generality. In one embodiment, a drop-out layer is used to prevent overfitting while combining the feature maps, resulting in a final feature vector. In one embodiment, the next stage of the network is two fully connected layers, which are used to classify the feature vector generated by the previous layers. In one embodiment, a final fully connected layer contains two neurons where one neuron represents the classification of the pixel as belonging to the vessel class, and other represents the non-vessel class.

The deep convolutional neural network is trained using the original OCT-A images along with the corresponding ground truth segmentation as inputs. A plurality of ground truth labeled vessel and non-vessel pixels are required for training. In one embodiment, data generated from a single expert are used to train the neural network. In another embodiment, data generated from multiple experts are used to train the neural network.

In one embodiment, training is performed using a 61×61 pixel square window around the training pixel. The size of the window around the training pixel may be varied without loss of generality. In one embodiment, the missing pixels at the borders of the training window are set to zero.

The trained network is then used to segment new OCT-A images not used for training. A square window of the same size used for the training purposes is extracted around each pixel of the image to be segmented. At the output of the deep convolutional neural network are two neurons, calculating the probably that the pixel belongs to a vessel class, and the probability that it belongs to the background class. For the pixels classified as vessels, each output pixel is assigned a grayscale value, with higher values representing higher confidence of the pixel being a vessel pixel. The output pixel values are aggregated into the output grayscale images. In one embodiment, the output image may be filtered with a small window, for example median filtered with a 3×3 window, in order to decrease the noise level in the image. The type of filter and size of the window may be varied without loss of generality. A threshold may be applied to the pixels classified as vessels in order to generate an image comprising of only the highest confidences. In one embodiment, the threshold is applied in the range from 0.6 to 0.9

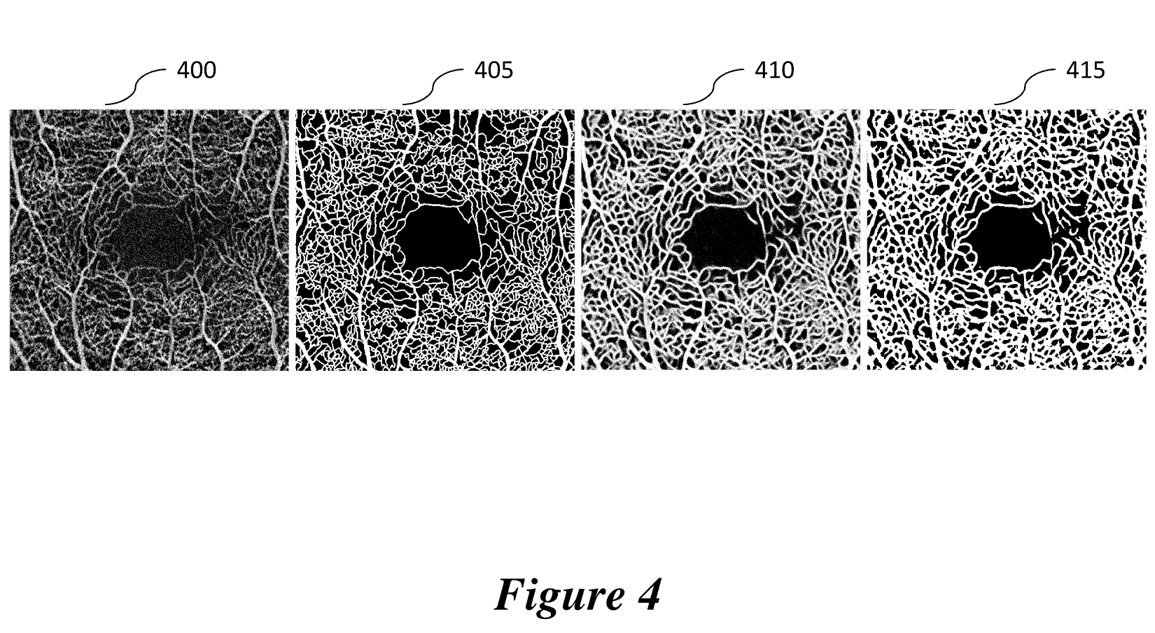

FIG. 4 presents an example of an original image (400), manual segmentation (405), the automated segmentation performed by an embodiment of the invention (410), and the output after thresholding (415). In the example, the pixels belonging to vessels in the original image are much brighter than the background pixels in the output image.

The performance of the deep convolutional neural network described herein as an example is described below. For the cross-validation and training of this embodiment, Rater A segmented all 80 OCT-A images. For the repeatability analysis, 10 images from this set were segmented at a second time by Rater A twice to evaluate intra-rater agreement; the first segmentation, used as the ground truth, is denoted Rater A1, and the repeat segmentation denoted Rater A2. These 10 images were additionally segmented by a different expert, Rater B, for assessing the intra-rater (different raters) agreement.

The segmentation performance was evaluated by pixel-wise comparison of the manually segmented images and the output of the deep convolutional neural network. Since one output of the deep convolutional network is a confidence level of a pixel belonging to the vessel class, the outputs were converted to images by applying a threshold, and binarizing the results. The performance was evaluated for different values of the threshold applied to the deep convolutional neural network output.

The number of true positives (TP), false positives (FP), false negatives (FN) and true negatives (TN) were calculated using pixel-wise comparison between a ground truth manual segmentation and a target, which was either another manual segmentation (from the same rater at a different time, i.e., Rater A1; or from a different rater, i.e., Rater B), or the output of the deep convolutional neural network. In this context, a pixel is considered as TP if it is marked as a blood vessel in both the ground truth manual segmentation and in the target. A pixel is considered as FN if it is marked as blood vessel in the ground truth manual segmentation but missed by the target. A pixel is considered as FP if it is marked as vessel by the target segmentation but it is not marked as blood vessel in the ground truth segmentation. A pixel is considered as TN if it is not marked as blood vessel in both the ground truth manual segmentation and in the target. Using the TP, FP, FN and TN the accuracy, the following values can be calculated: accuracy=(TP+TN)/(TP+TN+FP+FN); sensitivity=TP/(TP+FN); specificity=TN/(TN+FP) and positive predictive value (PPV)=TP/(TP+FP). Using the positive predictive value and sensitivity, the F1 measure is calculated using F1=2*Sensitivity*PPV/(Sensitivity+PPV)

For convenience, in this example, the accuracy measures discussed above and the original segmentation by Rater A (denoted Rater A1) was used as the ground-truth in order to assess its agreement with i) the repeat segmentation of Rater A (denoted Rater A2), ii) Rater B, and iii) the thresholded output of the deep convolutional neural network using an embodiment of the invention as presented in FIG. 3.

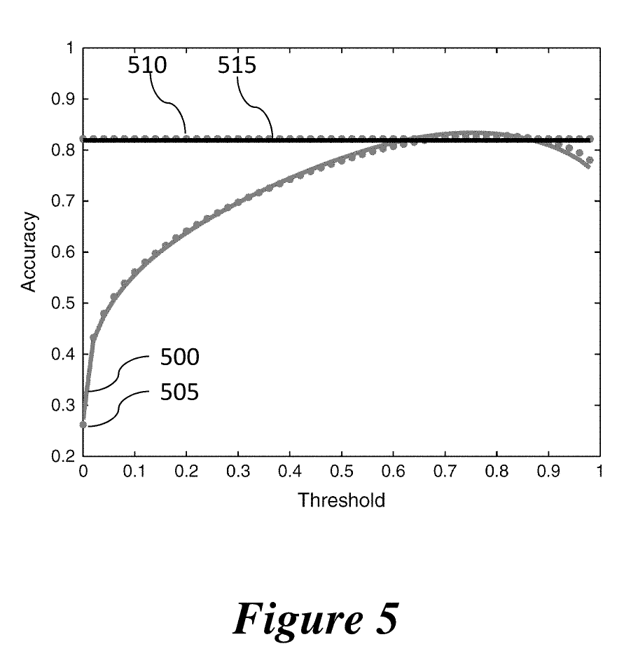

All of these measures can be calculated on individual images but can also be calculated for the whole dataset. In FIG. 5, the dotted curve (505) shows the accuracy for all the pixels in the dataset against a threshold value used to binarize the output of the network. The solid curve (500) is the accuracy using only the images used for assessing the inter-rater and intra-rater accuracies. The accuracy of blood vessel detection increases from the threshold value at 0, peaks at 0.8291 with threshold value of 0.78, and then begins to decline. When selecting a threshold to binarize the neural network output images, it is important to note that similar results are obtained in a wide range of thresholds, which indicates that the performance is not sensitive to the threshold chosen. The solid (515) and dotted lines (510) correspond to the intra-rater and inter-rater accuracies, respectively; the human raters perform only binary classification (vessel or not a vessel). The intra- and inter-rater accuracies for the manual raters are plotted as lines because they are independent of the threshold used for the machine based segmentation. From the figure, the intra-rater, inter-rater, and machine-rater accuracies are comparable, suggesting that the automated segmentation is comparable to that of a human rater. As it was expected, the accuracy of the repeated segmentation is better than the accuracy of the second rater but the difference is very small.

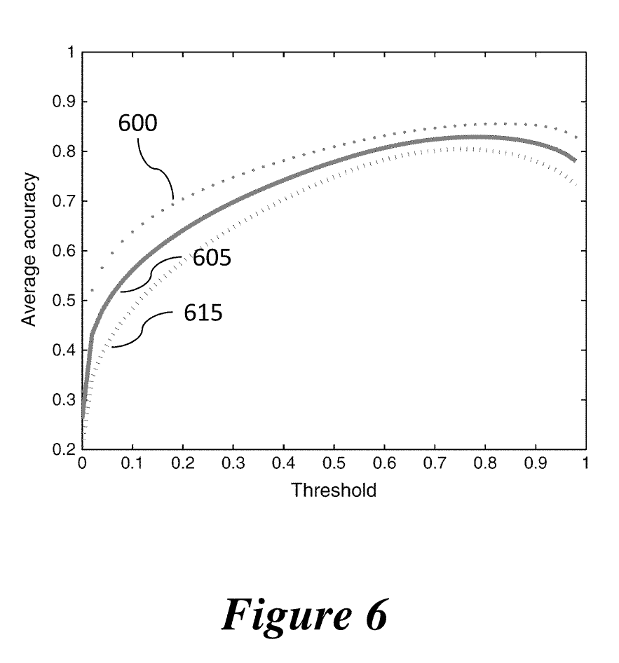

In FIG. 6, the mean accuracy of the segmentation by the deep convolutional neural network was calculated and averaged over all images (605). One standard deviation below the mean values is marked with a dashed curve (615) and one standard deviation above the mean values is marked with a dotted line (600). Qualitatively, the deviation of accuracies is reasonably small for different thresholds, with the maximum mean accuracy of 0.8337±0.0177 at the threshold value of 0.76, signifying that the performance of the methods is consistent over the whole dataset.

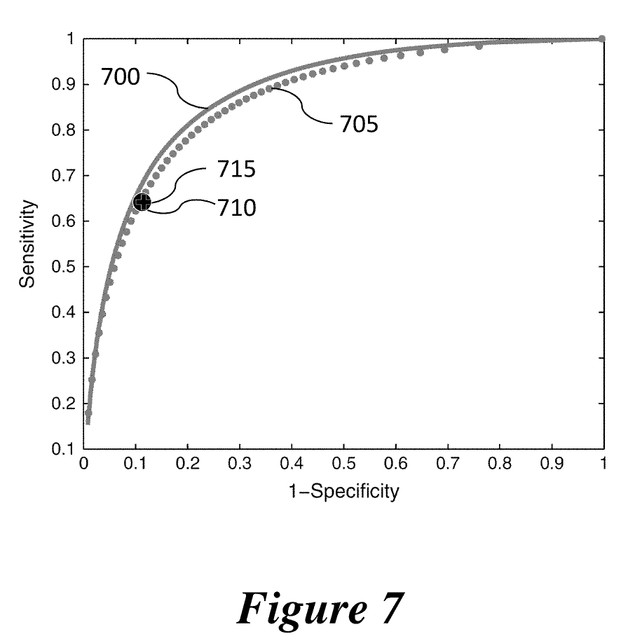

Using the sensitivity and specificity measurements over the range of thresholds the receiver operator characteristic (ROC) was plotted for the case of this example, as shown in FIG. 7. For this comparison, the segmentation by Rater A1 was taken to be the ground truth. The dotted curve (705) is the ROC curve for all pixels in the dataset and the solid curve (700) is the ROC curve for the images used for assessing the intra-rater and inter-rater accuracies, respectively. The ROC curve of the automated segmentation is compared to Rater A1. In the same figure, the point identified by the filled in circle (710) represents the sensitivity and specificity pair for Rater A2 compared to Rater A1 and the point identified by the cross (715) represents the sensitivity and specificity pair for Rater B compared to Rater A1.

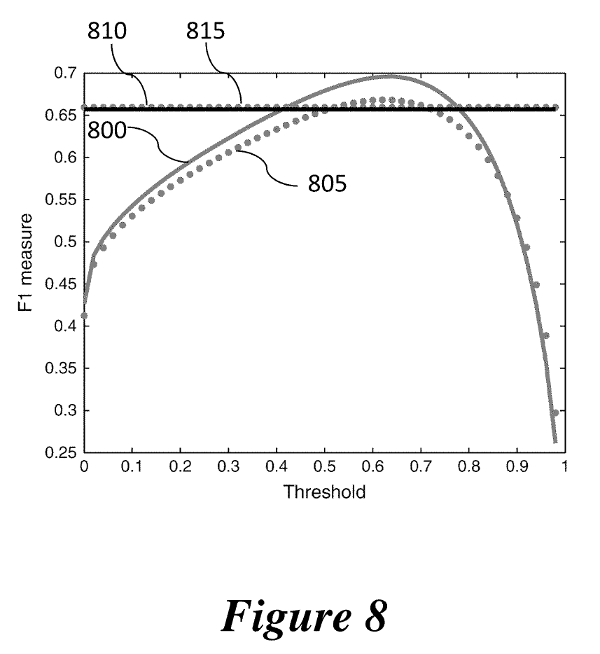

In FIG. 8, the F1 measure was calculated for the machine output using all the pixels from the dataset and shown with the dotted curve (805). The solid curve (800) is the F1 measure of the subset of images used for assessing the intra-rater and inter-rater accuracies. The solid line (815) and dotted line (810) are the intra-rater and inter-rater F1 measures, respectively. The performance of the deep convolutional neural network is demonstrated to be comparable to the performance of the expert human raters used to generate the ground truth. The F1 measure measures the trade-off between precision and recall (sensitivity) with each variable weighted equally. As such a higher F1-measure has a better balance between precision and recall. As can be observed in FIG. 8, there is a wide range of thresholds in which the balance between precision and recall is higher with the deep convolutional neural network than for the manual raters.

The accuracy percentage of one embodiment of the trained network was in the 80's (see FIG. 5 and FIG. 6). However, the performance of a machine learning based approach is closely linked to the quality of the training data. In the intra- and inter-rater comparison, there are similar degrees of agreement for the repeated segmentations by a single rater, and segmentations from two different raters, showing substantial intra- and inter-rater variability in the manual segmentation (see FIG. 7 and FIG. 8). This suggests that the trained network may perform as well as a human rater, but the performance of a human rater, the ground-truth for training the network, is limited due to the difficulty in delineating the capillaries. This in turn is related to the contrast, presence of motion artifact, and noise levels of the images. Hence, increasing the quality of the angiography images at the acquisition stage would increase the quality of the manual rater accuracy and repeatability. This in turn can reduce the noise level in the ground truth data and make the automated method more robust. The performance of the deep convolutional network can be further improved by producing a ground-truth that is measurably better than data from a single expert by using images segmented by two or more trained volunteers as the input to the learning procedure. A drawback to this approach would be the human labor cost of several trained raters segmenting a sufficiently large number of images for training purposes.

The problem of blood vessel segmentation in OCT-A images is challenging due to the low contrast and high noise levels in OCT-A images. We have presented a deep convolutional neural network-based segmentation method and validation using 80 foveal OCT-A images. From the results, the performance of the machine based segmentation was comparable to the performance of the manual segmentation by human raters. Given the amount of time (on the order of an hour) required for a human rater to perform a careful segmentation manually versus 2 minutes for the automated method, this represents a tool that could be useful in the clinical environment. The 2-minutes-procesing time could be improved by optimizing the neural network parameters and its implementation.

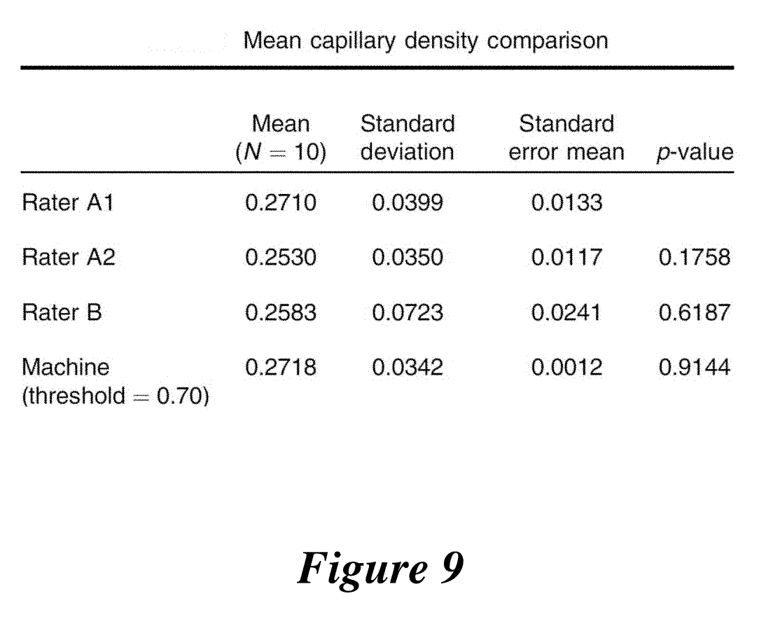

In addition to comparison with manual segmentation, the validity and merit of automated segmentation of medical images can be assessed using clinical parameters such as capillary density. This approach is particularly appropriate if the quality of the derived parameters can be measured, for example, by the correlation to other relevant clinical features, and if the quality of the manual segmentation ground truth is not reliable. Capillary density (CD) is a clinical measure of quantifying retinal capillaries present in the OCT-A images. In one embodiment, after segmentation of the vessels, the CD can be calculated as the number of pixels in the segmented areas. In our experiment, the CD was measured for each of the 10 images segmented by Rater A1, Rater A2, Rater B, and the network. The mean capillary density was calculated in order to evaluate the intra-rater, inte-rrater, and machine-to-rater repeatability of the CD measures. The table of results are presented in FIG. 9. A paired-samples t-test was conducted to compare the capillary density of manual and automated segmentations. As in above, Rater A1 was taken to be the ground truth used for comparison. There was no significant difference in the scores for either of the manual raters or the machine meaning that the segmentations from the network are comparable to a manual rater.

### INDUSTRIAL APPLICATIONS

OCDR, OCT, or OCT-A, or capillary-detection systems, and methods of this instant application is very useful for diagnosis and management of ophthalmic diseases such as retinal diseases and glaucoma etc. Instant innovative OCDR, OCT, or OCT-A, or vessels-detection diagnostic systems leverage advancements in cross technological platforms. This enables us to supply the global market a valuable automated blood-vessel imaging and detection tool, which would be accessible to general physicians, surgeons, intensive-care-unit-personnel, ophthalmologists, optometrists, and other health personnel.

This device can also be used for industrial metrology applications for detecting depth-dependent flow and micron-scale resolution thicknesses.

It is to be understood that the embodiments described herein can be implemented in hardware, software or a combination thereof. For a hardware implementation, the embodiments (or modules thereof) can be implemented within one or more application specific integrated circuits (ASICs), mixed signal circuits, digital signal processors (DSPs), digital signal processing devices (DSPDs), programmable logic devices (PLDs), field programmable gate arrays (FPGAs), processors, graphical processing units (GPU), controllers, micro-controllers, microprocessors and/or other electronic units designed to perform the functions described herein, or a combination thereof.

When the embodiments (or partial embodiments) are implemented in software, firmware, middleware or microcode, program code or code segments, they can be stored in a machine-readable medium (or a computer-readable medium), such as a storage component. A code segment can represent a procedure, a function, a subprogram, a program, a routine, a subroutine, a module, a software package, a class, or any combination of instructions, data structures, or program statements. A code segment can be coupled to another code segment or a hardware circuit by passing and/or receiving information, data, arguments, parameters, or memory contents.

What is claimed is:

1. A system, comprising:
a light source to emit a light to a beam splitter, which separates the light into two optical arms, a sample arm and a reference arm;
the sample arm further comprises of a sample and light delivery optics; and
a reference arm comprising a reference mirror;
a light returning from the sample and reference arms combined through the beam splitter and directed towards at least one detector to generate an optical interference signal;
an instrument controller for controlling the acquisition of the interference signal;
a processor to process the interference signal to generate at least one image;
manually segment at least one image to label features of interest;
train a neural network to extract the features of interest using the manually segmented images;
segment the features using the trained neural network.

2. System of claim 1; where the processor generates at least one of en face images, images comprising flowing material, and angiograms.

3. The system of claim 1; where the features of interest comprise of at least one of capillaries and vessels.

4. The system of claim 1; where the neural network is comprised of convolutional neural networks.

5. The system of claim 1; wherein the light source is a swept-source.

6. The system of claim 1; where the detection arm further comprises of a spectrometer.

7. A method, comprising of:
acquiring at least one image using an imaging device;
an expert manually segmenting the acquired image(s) to extract features of interest;
storing the manually segmented image(s) using a medium;
training a neural network to segment the features of interest using the manually segmented image(s);
acquiring a new image using the imaging device;
segmenting the new image using the trained neural network to extract the features of interest.

8. The method of claim 7; where the imaging device is an optical coherence tomography device.

9. The method of claim 7; where the imaging device is a common path interferometer.

10. The method of claim 7; where the features of interest comprise at least one of regions occupied by fluids, capillaries, retinal layers, choroidal layers, blood vessels, and lymph vessels.

11. The method of claim 7; where the experts comprise of at least one of clinicians, scientists and engineers.

12. The method of claim 7; where the neural network is implemented in hardware using at least one of FPGA, DSP and application-specific-integrated-circuits.

13. The method of claim 7; where the neural network is implemented in software using at least one of CPU, GPU, and RISC processors.

14. A system, comprising:
a light source to emit a light to a beam splitter, which separates the light into two optical arms, a sample arm and a reference arm;
the sample arm further comprises of a sample and light delivery optics; and
a reference arm comprising a reference mirror;
a light returning from the sample and reference arms combined through the beam splitter and directed towards at least one detector to generate an optical interference signal;
an instrument controller for controlling the acquisition of the interference signal;
a processor to process the interference signal to generate at least one image;
manually segment at least one image to label vessels;
using the manually segmented images to train a neural network to extract vessels;
segment new images using the trained neural network to extract vessels.

15. The system of claim 14; where the image is at least one of an en face image, an angiogram, and an en face angiogram.

16. The system of claim 15, where the angiogram is obtained using monitoring variations in at least one of image intensity and the phase of the optical interference signal.

17. The system of claim 14; wherein the light source is a swept-source.

18. The system of claim 14; wherein the light source is a broad-band source.

19. The system of claim 14; wherein the detector comprise of a spectrometer.

20. The system of claim 14; wherein a capillary density is computed using the new images with vessels extracted.
